(19) United States
(12) Patent Application Publication
(10) Pub. No.: US 2018/0087013 A1
(43) Pub. Date: Mar. 29, 2018

(54) Enzymic Saccharification Method of Biomass for Minimizing Generation of Metabolite of Contaminated Microorganisms, and Apparatus Therefor

(71) Applicant: Korea Research Institute of Chemical Technology, Daejeon (KR)

(72) Inventors: Ju-Hyun YU, Daejeon (KR); Young-Hoon HO, Seoul (KR); Gyeong-Tae EOM, Gyeonggi-do (KR); In-Yong EOM, Daejeon (KR)

(21) Appl. No.: 15/562,674

(22) PCT Filed: Nov. 30, 2015
(86) PCT No.: PCT/KR2015/012930

§ 371 Date: Sep. 28, 2017

(30) Foreign Application Priority Data
Apr. 9, 2015 (KR) 10-2015-0050318

(51) Int. Cl. C12M 1/34; C12P 7/10; C12P 19/00; C12M 1/00

(52) U.S. Cl. CPC C12M 1/34; C12P 7/10; C12M 21/12; C12P 19/00; C12M 41/26

(57) ABSTRACT

The present invention provides a method of terminating enzymatic saccharification by the early detection of organic acids produced by contamination by microorganisms contaminated in an enzymatic saccharification process of biomass or biomass pretreatment products; and a reactor therefor. The method of the present invention to maximize the saccharification rate and minimize metabolites secreted by microorganisms contaminated during the enzymatic saccharification of biomass or biomass pretreatment products and the reactor therefor to maximize the saccharification rate by converting the operating conditions of the enzymatic saccharification reactor to strongly inhibit microbial growth by early detection of the production of organic acids produced by contamination with microorganisms during the enzymatic saccharification of biomass and enable the manufacturing of biosugar containing almost no microbial metabolites.

### TECHNICAL FIELD

This application claims priority based on Korea Patent Application No. 10-2015-0050318, filed on Apr. 9, 2015, and all contents disclosed in the specification and drawings of that application are incorporated in this application.

The present invention relates to a method of enzymatic saccharification to minimize the metabolites produced by microorganisms contaminated during the saccharification of biomass to monosaccharides using a starch or cellulose hydrolase; and a reactor therefor. More specifically, a method of early detection to determine the point in time when the growth of unwanted microorganisms using as carbon sources the monosaccharides produced by the saccharification of biomass following the addition of an enzyme becomes significant, and by terminating the saccharification process to maximize the saccharification rate and minimize the production of microbial metabolites; and an reactor therefor.

### BACKGROUND ART

Since the reserves of fossil fuels such as petroleum and coal, those are being continuously consumed by mankind in daily life, are limited, and demand for fossil feels can fluctuate rapidly according to the economic cycle, the price of such fuels is often very unstable. Because rapid fluctuations in the prices of fossil fuels can have a huge effect on entire industries, there have been considerable efforts and investments to develop alternative energy resources, and to replace the consumption of fossil feels with renewable energy sources. An example of such a renewable energy source is bio-alcohol produced by using lignocellulosic biomass as the raw material. Bio-alcohol has already been produced commercially in many countries including the US and used as a transportation fuel. The types of lignocellulosic biomass used to produce bio-alcohol include corn stover, wheat straw, and sugar cane bagasse. Glucose is directly produced by the hydrolysis of cellulose, which is one of structural components in biomass.

In addition, algal biomass, including green algae and diatoms, have recently begun attracting attention as a third-generation biomass, and are currently being studied and developed for commercialization. Algal biomass contains not only carbohydrates, such as starch and cellulose, but also a large amount of oil, and is thus regarded as a promising bio-fuel resource for the synthesis of bioethanol and bio-diesel fuels.

The cellulose contained in lignocellulosic biomass is surrounded by other structural components, such as hemicelluloses and lignin. The complicated structure formed of these components enables the plants to stand firmly despite harsh weather conditions, such as rain and wind, prevents the infiltration of external water including rainwater into the tissues, and protects the plants from being extremely damaged by microbial infection. However, the same complicated plant structure is an obstacle that needs to be overcome when humans produce biochemical materials, such as bio-alcohol and bio-plastics, from plant biomass.

When converting the cellulose of biomass into glucose, the first step involves dissolving the hemicellulose or lignin to expose the cellulose. This process, called pretreatment of biomass, includes acid-catalyzed pretreatments and alkali-catalyzed pretreatment. After the pretreatment, the cellulose is converted to glucose by acid hydrolysis or enzymatic hydrolysis.

Due to the extremely harsh chemical reactions, in acid hydrolysis of cellulose, as a next step of biomass pretreatment, not only furfural and 5-hydroxymethyl-2-furaldehyde (HMF) are produced by the over-degradation of carbohydrates, but also phenolic materials are produced by the degradation of lignin. These degradation products are known to be major microbial inhibitors because they inhibit the growth of microorganisms, such as yeast, and reduce the yield of target microbial metabolites. Therefore, to use the glucose for microbial fermentation, the separation and purification of the glucose is required to get rid of these degradation products and impurities.

In contrast, when the enzyme is used to produce glucose from cellulose, it does not make the microbial inhibitors mentioned above, and thus is considered more appropriate for manufacturing a carbon source for microbial fermentation (hereinafter referred to as “fermentable sugar” or “biosugar”).

In the manufacturing of bioethanol or fermentable sugar by the pretreatment and enzymatic saccharification of lignocellulosic biomass, as the harshness of the chemical reactions involved in the pretreatment of biomass is increased, the conversion rate of cellulose to glucose is increased in the following enzymatic saccharification. For example, the harshness of wheat straw pretreatment is increased, the higher the concentration of dilute acid is mixed with the wheat straw, the higher the pretreatment temperature is applied, and the longer the high acid concentration and temperature are maintained for. As a result, hemicellulose and lignin are dissolved more, and thus the conversion of cellulose to glucose by enzymatic hydrolysis is increased in the following hydrolysis process. However, as the harshness of the pretreatment is increased, the microbial inhibitors mentioned above are significantly increased, and thus an additional detoxification process or a separation and purification process is required. On the other hand, if the biomass pretreatment process is so weak that it does not produce the microbial inhibitors, or if the microbial inhibitors are removed through washing with hot water after the harsh pretreatment, before the following conversion of cellulose to glucose by enzymatic saccharification, fermentable sugar can be manufactured even by a simple concentration process.

Among types of algal biomass, green algae and diatoms, which mostly containing starch or cellulose, do not have lignin in their body structure in contrast to lignocellulosic biomass. As a result, green algae and diatom do not require the high-temperature and high-pressure pretreatment used for lignocellulosic biomass, and the carbohydrates contained in their body, such as starch and cellulose, are easily converted to monosaccharides by amylase and cellulase.

The enzymatic saccharification of pretreated lignocellulosic biomass generally requires a long time, from 24 to 96 hours, while that of algal biomass requires from 12 hours to more than 72 hours. Lengthening the enzymatic saccharification time is advantageous to producing a large amount of glucose, because it only requires adding a small amount of expensive enzymes. However, safely protecting the glucose produced by the enzymatic hydrolysis of the pretreated biomass from other microorganisms for a long time is difficult. For example, Inbicon (Denmark), one of the leading companies in the development of technologies and devices for bioethanol production from lignocellulosic biomass, limits the concentration of lactic acid produced by contamination with microorganisms except yeast to 0.5% or less in the bioethanol manufacturing process, indicating that microbial contamination is very common in a saccharification or fermentation process.

To prevent microbial contamination in the enzymatic saccharification of biomass and ethanol fermentation, therefore, various methods have been proposed and employed. Common examples of such methods include sterilization of the biomass or biomass pretreatment products before the enzymatic saccharification, for example, by high-temperature steaming or the addition of acid, alkali, or ethanol, and the addition of an antibiotic, such as penicillin, or an inhibitor that is harmless to fermentation microorganisms including yeast, but which effectively inhibits the growth of other unnecessary microorganisms.

In addition, the prior arts for recovering various microbial inhibitors produced by the pretreatment process, and for introducing some of the recovered microbial inhibitors into the enzymatic saccharification process to inhibit unwanted microbial growth in a biomass-based bioethanol manufacturing process, are revealed in U.S. Pat. No. 8,187,849 and U.S. Pat. No. 8,496,980 registered by Inbicon, The prior arts for controlling the residues of microbial inhibitors by controlling the liquid recovery ratio in a solid-liquid separation process of the pretreatment products and for inhibiting unwanted microbial growth by using microbial inhibitors are revealed in Korean Patent No. 3449552 and Korean Patent No. 1504197 registered by the Korea Research Institute of Chemical Technology.

However, in contrast to the manufacturing of bioethanol, where relatively pure ethanol may be easily recovered by distillation, the inclusion of microbial metabolites that may be produced by microbial contamination, such as lactic acid, should be often avoided in the high-concentration fermentable sugars produced using biomass as a raw material, such as biosugar. In particular, the biosugar used for the manufacturing of polylactic acid (PLA), which is a bio-plastic that has already been commercialized and produced in large quantities, should not contain any type of unwanted optically different lactic acids.

Since the target biosugar to be produced is dissolved in water, its recovery cannot be earned out via simple distillation, as in the case of ethanol. Inevitably, an expensive process such as ion chromatography needs to be performed to remove the unwanted microbial metabolites. In addition, using an antibiotic to inhibit the growth of a specific microorganism is inappropriate for the manufacturing of general purpose-fermentable sugars.

The inventors of the present invention have learned through numerous experiments manufacturing biosugar that microbial growth is not always successfully inhibited by the prior arts as intended, when producing fermentable sugars for industrial applications with biomass as the raw material. In particular, while the enzymatic saccharification process used for manufacturing biosugar requires a long-time enzymatic reaction to reduce the amount of enzymes used, the growth of unwanted microorganisms usually began within 24 hours after the beginning of the saccharification, and their population was significantly increased after 48 hours, except in a few cases where signs of microbial contamination were not found 72 hours after the beginning of the saccharification.

In addition, lactic acid was commonly produced by the growth of unwanted microorganisms, and thus the consumption of an alkali to maintain constant acidity in the enzymatic saccharification process was also increased. Many studies have conducted for the microorganisms to separate from the contaminated samples and identified them to investigate their physiological properties, it showed that most of the microorganisms that thrived in the enzymatic hydrolysis at 50° C. were Bacillus coagulans. This microorganism well grows at around 50° C., may survive high-temperature sterilization by preparing spores, may grow well even in the presence of high concentration of furfural which inhibits the growth of various microorganisms, and, produces lactic acid and acetic acid as metabolites.

The growth of Bacillus coagulans, the representative microorganism contaminated, can be controlled by antibiotics including penicillin, but these kinds of microbial inhibitors are not applicable to the manufacture of a general-purpose fermentable sugar. And even though they might be used for, the increase of the production cost would be inevitable.

Rather than adding the microbial inhibitors produced in the pretreatment process to prevent the enzymatic saccharification process from the microbial contamination, early detecting the microbial contamination that rarely occurs during enzymatic saccharification, thereby, converting the enzymatic saccharification system to the conditions where microbial growth is strongly inhibited, is desirable to prevent the microbial growth and the production of their metabolites.

### Technical Problems

Therefore, the present invention relates to a method of minimizing the production of microbial metabolites and maximizing the enzymatic saccharification time during an enzymatic saccharification process by early detecting the microbial metabolites produced by microorganisms in the enzymatic saccharification of biomass or biomass pretreatment products and by promptly converting a saccharification reactor to conditions under which microbial growth is strongly inhibited; and an enzymatic saccharification reactor.

### Technical Solutions

The present invention provides a method by detecting the organic acid produced by the microorganisms contaminated in the enzymatic saccharification of the biomass or biomass pretreatment products, and converting an enzymatic saccharification reactor to conditions under which microbial growth is strongly inhibited; and a reactor therefor.

More specifically, the present invention provides an enzymatic saccharification method of biomass to minimize the production of microbial metabolites by microorganisms contaminated, wherein the enzymatic saccharification method of biomass includes: an enzymatic saccharification process of biomass or biomass pretreatment products; measuring the pH change by monitoring the pH of an enzymatic saccharification system during the enzymatic saccharification; adjusting the pH of the enzymatic saccharification system to a pH range suitable for cellulose hydrolase by injecting an aqueous acid or base into the enzymatic saccharification system; measuring the pH change rate by determining the time length of the enzymatic saccharification system to change from a re-adjusted value by injecting an aqueous alkali to a preset lower limit; detecting microbial contamination by comparing the measured value of the pH change rate to a previous measured pH change rate value, to detect the beginning of microbial contamination based on a time point when the pH measurement value starts to reach a specific pH value or a specific ratio; detecting a critical time point by comparing the measured pH change rate value to a previous measured pH change rate value and to detect the time point when the pH measurement value falls below a specific pH value or a specific ratio which is the critical time point when the microbial contamination is too severe to continue the enzymatic saccharification process; and immediately converting the enzymatic saccharification system to operate at the critical time point at the conditions under which microbial growth is strongly inhibited.

In addition, the present invention provides a enzymatic saccharification method of biomass to minimize the production of microbial metabolites by microorganisms contaminated, wherein the measuring the pH change of the enzymatic saccharification system of the present invention is within a range where the hydrolase activity is maintained.

In addition, the present invention provides an enzymatic saccharification method of biomass to minimize the production of microbial metabolites by microorganisms contaminated, wherein the change of pH in the enzymatic saccharification system process during the enzymatic saccharification of biomass begins with the production of an organic acid by the hydrolysis of hemicellulose.

In addition, the present invention provides an enzymatic saccharification method of biomass to minimize the production of microbial metabolites by microorganisms contaminated, wherein the change of pH in the enzymatic saccharification system during the biomass enzymatic saccharification process begins with the production of an organic acid by the growth of microorganisms existing in the enzymatic saccharification system.

In addition, the present invention provides an enzymatic saccharification method of biomass to minimize the production of microbial metabolites by microorganisms contaminated, wherein an injection interval of an aqueous alkali due to the pump operation is used instead of the measured rate of pH change to detect the microbial contamination during the enzymatic saccharification process.

In addition, the present invention provides an enzymatic saccharification method of biomass to minimize the production of microbial metabolites by microorganisms contaminated, wherein, in the detection of microbial contamination, the time taken by the pH of the enzymatic saccharification system to be lowered to a preset lower limits after a certain amount of an alkali is injected or the time interval between the point in time when the pH is increased by the operation of alkali pump and the point in time when the alkali pump is operated again is compared with previous measurements, and the point in time when microbial contamination substantially begins is determined to be the point in time when the present measurement becomes lower by a preset ratio in comparison with the previous measurements.

In addition, the present invention provides an enzymatic saccharification method of biomass to minimize the production of microbial metabolites by microorganisms contaminated, wherein, in the detection of a critical point in time, the time taken by the pH of the enzymatic saccharification system to be lowered and exceeds a preset lower limit after a certain amount of an alkali is injected or the time interval between the point in time when the pH is increased by the operation of alkali pump and the point in time when the alkali pump is operated again is compared with previous measurements, and the critical point in time when the microbial contamination is too severe to continue the enzymatic saccharification process is determined to be the point in time when the current measurement is further lowered and exceeds a preset ratio in comparison with the previous measurements.

In addition, the present invention provides an enzymatic saccharification method of biomass to minimize the production of microbial metabolites by microorganisms contaminated, wherein the critical point in time when the microbial contamination is too severe to continue the enzymatic saccharification process is determined to be the point in time when the total amount of an alkali injected after the detecting of microbial contamination to adjust the pH of the enzymatic saccharification system becomes equal to an organic acid equivalent allowed as a microbial metabolite.

In addition, the present invention provides an enzymatic saccharification method of biomass to minimize the production of microbial metabolites by microorganisms contaminated, wherein, at the critical point in time when the microbial contamination is too severe to continue the enzymatic saccharification, the operating conditions of the enzymatic saccharification system are rapidly converted to the conditions under which microbial growth is strongly inhibited by promptly adding an acidic or alkali to convert the pH to a level at which microorganisms may grow no longer or by rapidly cooling the enzymatic saccharification system to a temperature at which microorganisms may no longer grow.

More specifically, the present invention provides a biomass saccharification reactor to minimize the production of microbial metabolites by microorganisms contaminated, wherein the biomass saccharification reactor includes: an enzymatic saccharification reactor for the saccharification of biomass or biomass pretreatment products; a pH measurement device for measuring the pH of saccharification products in the enzymatic saccharification reactor; a pump for providing an alkali into the enzymatic saccharification reactor; a pH adjustment device for the enzymatic saccharification reactor by controlling the amount of an alkali injected by an alkali pump or the interval between injections based on the pH of the saccharification products measured by pH measurement device; and a thermostat for keeping the temperature of the saccharification reactor constant.

In addition, the present invention provides a biomass saccharification reactor to minimize the production of microbial metabolites by microorganisms contaminated, wherein the biomass saccharification reactor further includes a temperature adjustment device for rapidly cooling the enzymatic saccharification system.

### Advantageous Effects of Invention

The method of the present invention to maximize the saccharification rate and minimize microbial metabolites secreted by microorganisms contaminated in the enzymatic saccharification of biomass or biomass pretreatment products and the reactor therefor may maximize the saccharification rate by converting the operating conditions of the enzymatic saccharification reactor to strongly inhibit the microbial growth by the early detection of an organic acid produced by the unwanted microorganisms contaminated during the enzymatic saccharification of biomass, and enable the production of biosugar containing almost no microbial metabolites.

### BRIEF DESCRIPTION OF DRAWINGS

FIG. 1 is a schematic diagram showing the enzymatic saccharification method of biomass to minimize the microbial metabolites produced by microorganisms contaminated according to one embodiment of the present invention.

FIG. 2 is a flow chart showing the method of terminating enzymatic saccharification by the early detection of an organic acid produced by the contamination of unwanted microorganisms in the enzymatic saccharification process of biomass or biomass pretreatment products according to one embodiment of the present invention.

FIG. 3 is a schematic diagram showing the biomass saccharification reactor according to one embodiment of the present invention.

### MODES FOR CARRYING OUT INVENTION

The present invention provides an enzymatic saccharification method of biomass to minimize the production of microbial metabolites by microorganisms contaminated, wherein tire enzymatic saccharification method of biomass includes: an enzymatic saccharification process of biomass or biomass pretreatment products; measuring the pH change by monitoring the pH of an enzymatic saccharification system during the enzymatic saccharification; adjusting the pH of the enzymatic saccharification system to a pH range suitable for cellulose hydrolase by injecting an aqueous acid or base into the enzymatic saccharification system; measuring the pH change rate by determining the time length of the enzymatic saccharification system to change from a re-adjusted value by injecting an aqueous alkali to a preset lower limit; detecting microbial contamination by comparing the measured value of the pH change rate to a previous measured pH change rate value, to detect the beginning of microbial contamination based on a time point when the pH measurement value starts to reach a specific pH value or a specific ratio; detecting a critical time point by comparing the measured pH change rate value to a previous measured pH change rate value and to detect the time point when the pH measurement value falls below a specific pH value or a specific ratio which is the critical time point when the microbial contamination is too severe to continue the enzymatic saccharification process; and immediately converting the enzymatic saccharification system to operate at the critical time point at the conditions under which microbial growth is strongly inhibited.

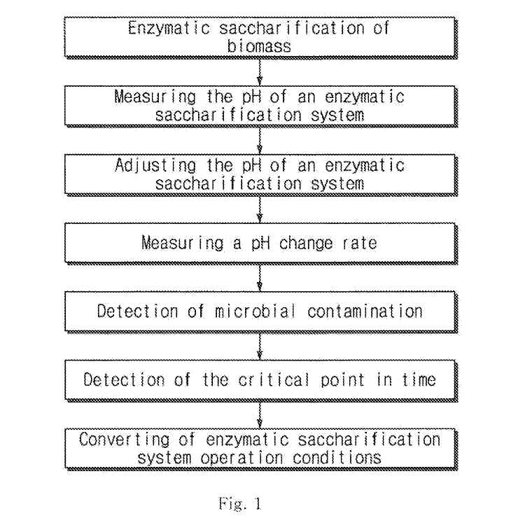

FIG. 1 is a schematic diagram of the process for the enzymatic saccharification method of biomass to minimize the microbial metabolites produced by microorganisms contaminated.

More specifically, to maximize the sugar yield by terminating the enzymatic saccharification by early detecting the contamination produced by microorganisms secreting organic acids, such as lactic acid, in the enzymatic saccharification of biomass or biomass pretreatment products, and to minimize the contamination of biosugar with the microbial metabolites, the prevent invention provides a method of, 1) detecting a point in time when the microbial contamination occurs by directly and indirectly measuring the pH of the enzymatic saccharification system and comparing it to detect an increase in the rate of pH change of 10% to over 50% in comparison with the slowest pH change rate; 2-1) by detecting the critical point in time when the microbial contamination is too severe to continue the enzymatic saccharification process by directly and indirectly measuring the pH of the enzymatic saccharification system and comparing the measured pH to detect an increase in the rate of pH change of 70% to over 90% in comparison with a slowest pH change rate; and 3) for rapidly converting the enzymatic saccharification system to the operating conditions to prevent microbial growth at the critical point.

In addition, the present invention provides a method of 2-2) detecting the critical point in time when the microbial contamination is too severe to continue the enzymatic saccharification process as the point in time when the total amount of an alkali injected to adjust the pH of the enzymatic saccharification system becomes equal to an organic acid equivalent allowed as a microbial metabolite, after the detection of the microbial contamination point in time detected by an increase in the rate of the pH change by 10% to over 50% in comparison with the slowest pH change rate by directly and indirectly measuring the pH of the enzymatic saccharification system and comparing the pH measurements.

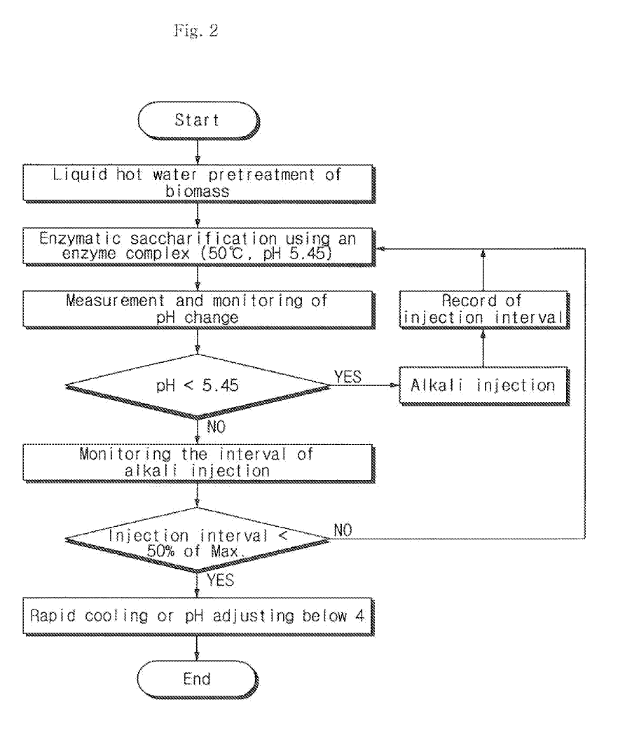

FIG. 2 is a flow chart showing the method of early detecting organic acids produced by the microbial contamination in the enzymatic saccharification process of biomass or biomass pretreatment products, thereby, terminating enzymatic saccharification according to one embodiment of the present invention, wherein the biomass is pretreated with liquid hot water to form a biomass pretreatment product, which undergoes saccharification through hydrolysis performed by using enzyme complex at about 50° C. and pH 5.45. The pH change of the enzymatic saccharification system where the biomass saccharification occurs is measured and monitored. When the pH of the enzymatic saccharification system becomes lower than 5.45, which is out of the pH range where the hydrolase activity is maximized, a quantified amount of an alkali is added to keep the pH in the range where the hydrolase activity is maximized, and then the interval between the alkali injections is monitored while continuing the saccharification. During the biomass enzymatic saccharification of the present invention, a first change in pH occurs with the production of acetic acid, an organic acid, released by the hemicellulose hydrolysis. As the enzymatic saccharification continues, the pH change rate is gradually decreased by the consumption of the acetic acid produced from hemicellulose. Subsequently, with the growth of the microorganisms in the enzymatic saccharification system, a second change in pH occurs due to the production of lactic acid, which is another organic acid. During the enzymatic saccharification, the pH change of the enzymatic saccharification system is measured and monitored, and the point in time when the alkali injection interval is less than 50% of the longest injection interval in the saccharification process is detected to be the point in time when an organic acid begins to be rapidly produced by the microbial contamination. At the point in time, the saccharification process is terminated by rapidly cooling the saccharification reactor to a temperature equal to or lower than 10° C. or by adjusting the pH to be equal to or lower than 4.

The biomass or biomass pretreatment products undergoing enzymatic saccharification in the present invention are biomass or biomass pretreatment products containing at least one starch or cellulose that can be used for producing glucose as a result of enzymatic saccharification, for example, agricultural by-products including corn stover, sunflower stalks, empty fruit bunches of oil palm, and palm trunk, energy crops including miscanthus and reed, lignocellulosic biomass including eucalyptus, acacia, willow, and hybrid poplar, and algal biomass including green algae such as chlorella and diatoms, but not limited thereto.

In the present invention, the biomass may undergo enzymatic saccharification without pretreatment, or may be pretreated as a substrate of the enzymatic saccharification to increase the efficiency of enzymatic saccharification by acid catalyzed pretreatment including liquid hot water pretreatment and dilute acid pretreatment, alkali catalyzed pretreatment using sodium hydroxide, calcium hydroxide, or ammonia. In addition, the biomass may be used as a substrate of the enzymatic saccharification by undergoing high-temperature sterilization to sterilize the microorganisms before the saccharification process.

In the present invention, the enzymes used for the biomass saccharification process are not particularly limited to specific enzymes because the enzymes are dependent on the types of biomass. Generally, an amylase complex may be used for the saccharification of starch, an enzyme complex including cellulase, hemicellulase, and pectinase may be used for the saccharification of cellulose and hemicellulose, and an amylase-cellulase complex may be used for the saccharification of biomass that includes both starch and cellulose. The enzymes have an optimal range of temperature and pH for biomass saccharification. For example, the optimal temperature and pH for Cellic CTec2 (Novozymes), which is a cellulose hydrolase, are 45 to 55° C. and 4.5 to 5.5, respectively, and those for Celluclast 1.5 L (Novozymes) are 45 to 55° C. and 4.5 to 5.2, respectively.

In the present invention, a variable that is used for the early detection of microbial contamination during the enzymatic saccharification of biomass or biomass pretreatment products is the pH of the enzymatic saccharification system, which is changed by the secretion of an organic acid by microorganisms. The enzymatic saccharification system is gradually acidified as organic acids including lactic acid and acetic acid are secreted by the growth of the microorganisms, such as Bacillus coagulans, that are not completely sterilized even at a high temperature, by forming spores in the biomass or the microorganisms that are commonly found in the environment and thus may be introduced into the enzymatic saccharification system with air during the enzymatic saccharification process, The acidification of the enzymatic saccharification system is described below in detail using the example of the enzymatic saccharification of a lignocellulosic biomass pretreatment product, performed by using a cellulase complex.

First, corn stover is hydrated by added water, and pretreated with liquid hot water at 190° C. for 20 minutes, and added to a batch fermenter for enzymatic saccharification. Cellic CTec2, a cellulose hydrolase complex, is added to the batch fermenter, which is kept at 50° C. and pH 5.45 and stirred, and monosaccharides, such as glucose and xylose, are produced by the hydrolysis of cellulose and hemicellulose. At the same time, the enzymatic saccharification system is gradually acidified by acetic acid attached to the xylan backborn as a functional group of the hemicellulose by an ester bond but released by hydrolysis. As the pH of the enzymatic saccharification system is gradually lowered below 5.45, the alkali pump is operated to inject a certain amount of an alkali to neutralize the acetic acid and thus increase the pH to over 5.45.

With time, as the acetic acid remaining in hemicellulose is consumed, the pH change in the enzymatic saccharification system becomes extremely slow. Meanwhile, within 24 to 48 hours in the enzymatic saccharification system, as the concentration of glucose increases and the spores of Bacillus species or Lactobacillus species that have survived in the biomass or that have been introduced to the enzymatic saccharification system with air are germinated and begin to grow exponentially, and lactic acid starts to be secreted into the saccharification products, although the starting time is not constant.

From that time on, the pH change of the enzymatic saccharification system, which was decreasing before that time, begins to increase. In other words, the lowering rate of pH from a pH value higher than 5.45 to a pH value lower than 5.45 is increased, or the time interval between the alkali pump operation to increase the pH from a value lower than 5.45 to a value higher than 5.45 is shortened. These two changes may be electronically detected. In the present invention, the start of microbial contamination is determined by an increase in the rate of pH change above an arbitrarily preset ratio within a range of 20% to 50% of the slowest change rate or by a reduction in the alkali pump operating interval below an arbitrarily preset ratio within a range of at least 20% to 50% of the longest pump operation interval.

As the enzymatic saccharification continues, the critical point in time when the microbial contamination is too severe to continue the enzymatic saccharification process is determined to be an increase in the pH change rate over an arbitrarily preset ratio within a range of at least 70% to 90% of the slowest measured change rate or as a decrease in the operating interval of an alkali pump below an arbitrarily preset ratio within a range of at least 70% to 90% of the longest pump operating interval.

At the critical point in time, the temperature of the enzymatic saccharification system is promptly reduced to below 10° C. to prevent the growth of microorganisms contaminated. Therefore, as a method for the early detection of contamination by microorganisms secreting organic acids including lactic acid during the enzymatic saccharification of biomass or biomass pretreatment products, the present invention provides a technology for electronically determining the points in time when the rate of change in pH in the enzymatic saccharification system is increased by a preset ratio in comparison with the slowest measured change in pH and when the operating interval of the alkali pump is reduced by a preset ratio in comparison with a longest measured interval.

In addition, the present invention provides a method of maximizing the saccharification rate and the saccharification yield by rapidly converting the operating conditions of the enzymatic saccharification system to conditions where the microbial growth contaminated is significantly inhibited at a point in time when contamination by microorganisms secreting organic acids, such as lactic acid and acetic acid, is severe during the enzymatic saccharification of biomass or biomass pretreatment products. The operating conditions of the saccharification reactor may be converted to operating conditions that enable a rapid inhibition of the growth of the microorganisms contaminated by cooling the inside of the saccharification reactor to a temperature of about 10° C. or by decreasing the pH below 4, and cooling the saccharification reactor when appropriate, considering the subsequent treatment of the saccharification products.

The present invention may be applied to the enzymatic saccharification of biomass for the manufacturing of biosugar to maximize the saccharification rate and to prevent the deterioration of biosugar by easily detecting the production of acidic metabolites, such as lactic acid, to minimize microbial contamination without performing a chemical analysis, by taking samples at certain intervals and using an analytical instrument, such as high-performance liquid chromatography (HPLC).

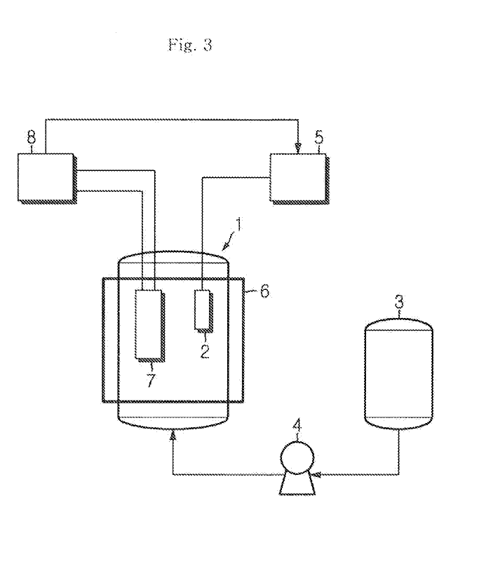

FIG. 3 is a schematic diagram showing the biomass saccharification reactor according to one embodiment of the present invention, wherein the biomass saccharification reactor to minimize the production of metabolites by microorganisms contaminated may include: (1) an enzymatic saccharification reactor tor the saccharification of biomass or biomass pretreatment products; (2) a pH measurement device of saccharification product for measuring the pH of saccharification products in the enzymatic saccharification reactor; (3) an alkali and (4) a pump to provide an alkali into the enzymatic saccharification reactor; (5) an pH adjustment device of enzymatic saccharification reactor for adjusting the pH in the enzymatic saccharification reactor by controlling the amount of an alkali injected by an alkali pump, or the injection interval, according to the pH of the saccharification products measured by the pH measurement device of saccharification product; (6) a thermostat for keeping the temperature of the saccharification reactor constant; (7) a temperature adjustment device for rapid cooling of the saccharification reactor; and (8) a controlling device to control the entire biomass saccharification reactor.

The enzymatic saccharification method of biomass to minimize the production of metabolites by microorganisms contaminated may be applied to the enzymatic saccharification of biomass or biomass pretreatment products to maximize the saccharification rate and to minimize the contamination of the saccharification solution by microbial metabolites by early detection of the contamination produced by microorganisms secreting an organic acid, such as lactic acid, and the termination of the enzymatic saccharification by: 1) detecting the point in time of the occurrence of the microbial contamination by directly and indirectly measuring the pH of the enzymatic saccharification system and comparing the pH measurements to detect an increase in the rate of pH change by 10% to over 50% in comparison with the slowest pH change rate; 2-1) detecting the critical point in time when the microbial contamination is too severe to continue the enzymatic saccharification process by directly and indirectly measuring the pH of the enzymatic saccharification system and comparing the measured pH to detect an increase in rate of the pH change from 70% to over 90% in comparison with the slowest pH change rate; and 3) rapidly converting the operation conditions of the enzymatic saccharification system at the critical point in time to prevent microbial growth, wherein the operating conditions of the saccharification reactor may be rapidly converted to operating conditions enabling the rapid inhibition of microbial growth by cooling the inside of the saccharification reactor to a temperature of about 10° C. by using the temperature adjustment device, or by decreasing the pH below 4 by using the pH adjustment device, and cooling with the saccharification reactor when appropriate, considering the subsequent treatment of the saccharification products.

Hereinafter, the present invention will be described in further detail with reference to examples. These examples are for illustrative purposes only and are not to be construed to limit the scope of the present invention.

### Example 1

### Preparation of Microbial Contamination Detector Based on the Operating Time Interval of the Alkali Pump

A microbial contamination detector was prepared, wherein the microbial contamination detector includes a control panel for measuring the operating time interval by sensing the electric current flowing through an alkali pump of the saccharification reactor; a monitoring program (LabView) showing the operating state of the detector; and a personal computer for executing the monitoring program. The detector was attached to a fermenter (7 L fermenter, Biotron, Hanil Scientific Inc., Seoul) and connected to the internet-through a LAN line. The microbial contamination detector was programed to measure the time interval of the alkali pump of the saccharification reactor, to detect the point in time when a newly detected time interval was below an arbitrarily preset ratio (for example, 50%) in comparison with the longest time interval, consecutively repeated for three times or more. This will notify a manager of the reactor of the point in time via an alarm and a mobile phone text message over the internet, and cool the saccharification reactor to a temperature below 10° C.

### Example 2

### Enzymatic Saccharification of Empty Fruit Bunch of Oil Palm by Using the Critical Point in Time Detection Function of the Microbial Contamination Detector

A pulverized empty fruit bunches of oil palm (20 mesh or under, Indonesia, Korindo Group) of 680 g was mixed with 8 L of distilled water, and the mixture was kept at room temperature overnight. The resulting sample was heated in a 10 L high pressure reactor (Hanwool Engineering, Seoul) to 191° C. and kept at that temperature for 15 minutes for pretreatment. Then, the sample was rapidly cooled, and divided between two cotton cloth-sacks. The sample was then dehydrated by using a spin dryer (Hanil Electric, Seoul) for one hour to prepare a solid substrate for enzymatic saccharification. The solid substrate for enzymatic saccharification was prepared by repeating the pretreatment and its dehydration 14 times. Distilled water (500 ml) was added to a saccharification reactor jar, and 68 ml of Cellic CTec2 (Novozymes Korea, Seoul) was added as a saccharification enzyme.

While the saccharification reactor was operated at 50° C., pH 5.45, and at a stirring rate of 100 rpm, an equivalent amount of the 680 g (dry weight) pretreatment product was put into the saccharification reactor by dividing three times. An alkali pump was connected to an aqueous sodium hydroxide (2 %, w/v). The microbial contamination detector in Example 1 was programmed to measure the time interval of the operation of the alkali pump, to detect the point in time when a newly detected time interval was below 50% in comparison with the longest time interval, and this interval was repeated consecutively three times or more. Then a reactor manager would be notified of the point in time over the internet via a mobile phone text message with an alarm, and to cool the saccharification reactor to a temperature below 10° C. After initiating the saccharification, the saccharification process was continued by operating the entire reactor until the alarm was given. Immediately after the alarm or the text message notification, the saccharification reactor was rapidly cooled to a temperature of around 10° C., and samples were taken to measure the sugar concentration and the lactic acid concentration with an HPLC (Waters, US). The same experiment was performed two more times, and the results are shown hi Table 1.

### Example 3

### Enzymatic Saccharification of Empty Fruit Bunches of Oil Palm Using the Alkali Injection Limiting Function of the Microbial Contamination Detector

In Example 3, to maintain the lactic acid content to below 0.5% of the glucose content following enzymatic saccharification, when the point in time of microbial contamination in the saccharification system was detected, a saccharification termination point in time was determined, based on the amount of an alkali injected to neutralize lactic acid.

1,000 ml distilled water was added to a saccharification container, and 100 ml of Cellic CTec2 (Novozymes Korea, Seoul) was added as a saccharification enzyme. The alkali pump was connected to a 2% sodium hydroxide. While the saccharification reactor was operated at 50° C., pH 5.45, and a stirring rate of 100 rpm, an equivalent amount of the 1,000 g (dry weight) pretreatment product of the empty fruit bunches of oil palm was put into the saccharification reactor jar by dividing four times. The microbial contamination detector in Example 1 was programed to measure the time interval of the alkali pump operation to detect a point in time as a substantial microbial contamination when the interval of alkali pump operation decreased to below 50% in comparison with the longest time interval three times or more in a raw. This substantial microbial contamination was used to detect a critical point in time when the cumulative amount of alkali injection becomes 33.4 ml, and to notify a reactor manager of the detected critical time point by using a mobile phone text message through the internet with an alarm, and to cool the saccharification reactor to a temperature below 10° C.

After initiating the saccharification, the saccharification process was continued by operating the entire reactor until the alarm was given. Immediately after the alarm or the text message notification, the saccharification reactor was rapidly cooled to a temperature of around 10° C., and samples were taken to measure the sugar concentration and the lactic acid concentration by using an HPLC (Waters, US). The same experiment was performed two more times, and the results are shown in Table 1.

### Comparative Example 1

### Enzymatic Saccharification of Empty Fruit Bunches of Oil Palm Using Antibiotics

An experiment similar to Example 2 was performed, by using pulverized dried empty fruit bunches of oil palm as a raw material (20 mesh or under, Indonesia, Korindo Group). For the experiment, an aqueous penicillin/streptomycin solution (Sigma, Product No. P4333-100ML) was added as an antibiotic to inhibit microbial growth at a ratio of 1 μl per 1 ml of the saccharification product. During 120 hours saccharification, a small amount of samples were taken at intervals of 24 hours to measure the sugar and lactic acid concentration, using an HPLC (Waters, US). Table 1 shows the saccharification rate, comparing the amount, of cellulose contained in the empty fruit bunches of oil palm converted into the equivalent amount of glucose.

### Comparative Example 2

### Enzymatic Saccharification of Empty Fruit Bunches of Oil Palm Without Antibiotics

An experiment similar to Example 2 was performed by .using pulverized empty fruit bunches of oil palm as a raw material (20 mesh or under, Indonesia, Korindo Group). No antibiotics was used in the experiment. During 48 hours saccharification, a small amount of saC (Waters, US). Table 1 shows the measurement results.

TABLE 1

| | Glucose and lactic acid yields in comparison with cellulose content (%) | | | | | | | |
|---|---|---|---|---|---|---|---|---|
| Item | Example | Example | Example | Example | Example | Example | Comparative | Comparative |
| Experiment | 2-1 | 2-2 | 2-3 | 3-1 | 3-2 | 3-3 | Example 1 | Example 2 |
| Saccharification | 61.0 | 48.3 | 53.6 | 56.2 | 49.5 | 46.9 | 96 | 36 |
| duration | | | | | | | | |
| (hour) | | | | | | | | |
| Glucose | 83.2 | 81.1 | 81.9 | 82.7 | 81.3 | 80.1 | 85.2 | 43.9 |
| Lactic acid | 0.0 | 0.0 | 0.0 | 0.1 | 0.2 | 0.2 | 0.0 | 1.1 |

As shown in Table 1, in Comparative Example 1 where the saccharification was performed using antibiotics, the saccharification process continued without microbial contamination, and the saccharification yield was maximized in 96 hours. In contrast, in Comparative Example 2 where no antibiotic was used and the enzymatic saccharification system was contaminated by microorganisms producing lactic acid, because the saccharification termination time point was delayed, the saccharification rate drastically decreased and a large amount of lactic acid was produced due to the metabolism of glucose to lactic acid. However, in the enzymatic saccharification of the empty fruit bunches of oil palm performed using the enzymatic saccharification method of biomass to minimize the production of metabolites by contaminated microorganisms of the present invention and the reactor therefor, the lactic acid production caused by microbial contamination was minimized and the duration of saccharification was safely maintained, resulting in a saccharification yield of 81 % to 85%. Therefore, the result showed that the present invention is very useful to safely maximize the saccharification rate in the manufacturing of fermentable sugars when using biomass as a raw material.

On the other hand, in Examples 3-1 to 3-3 for the control of lactic acid production after enzymatic saccharification, glucose was produced by maintaining a lactic acid content of less than 0.5% of the sugar content. This result showed that the method and reactor of the present invention may be used to minimize or control the production of metabolites by microorganisms contaminated, to maximize the saccharification rate in the manufacturing of fermentable sugars when using biomass as a raw material.

### INDUSTRIAL APPLICABILITY

The method of the present invention to maximize the saccharification rate during the enzymatic saccharification of biomass or biomass pretreatment products and minimize the production of metabolites secreted by microorganisms contaminated and the reactor therefor are very useful for the manufacturing of fermentable sugars through the enzymatic saccharification of biomass.

1. An enzymatic saccharification method of biomass to minimize production of metabolites by contaminated microorganisms, wherein the enzymatic saccharification method of biomass comprises:
enzymatic saccharification processing for the saccharification of the biomass or biomass pretreatment products;
measuring pH of an enzymatic saccharification system to determine a change in the pH during the biomass enzymatic saccharification processing;
adjusting the pH of the enzymatic saccharification system to a range of pH that is active for hydrolase by injecting an acid or alkali into the enzymatic saccharification system;
detecting microbial contamination to detect a point in time of a beginning of microbial contamination, when a pH measurement value starts to fall below a specific pH value or a specific ratio;
detecting a critical point in time to detect a point in time when the pH measurement value is further reduced below a specific pH value or a specific ratio, marking the critical point in time when the microbial contamination is too severe to continue the enzymatic saccharification processing; and
converting operating conditions of the enzymatic saccharification system to immediately convert the system at the critical point in time to conditions where microbial growth is strongly inhibited;
wherein detecting the microbial contamination comprises at least one of (1) measuring a rate of pH change to determine an amount of time taken by the enzymatic saccharification system to change the pH to a preset lower limit after adjustment fay injection of an alkali and comparing the measured rate of pH change to a previous measured rate of pH change, and (2) using an interval between alkali injections due to operation of an alkali injection pump.

2. The enzymatic saccharification method of biomass to minimize the production of metabolites by the contaminated microorganisms of claim 1, wherein the change in the pH of the enzymatic saccharification system process during the biomass enzymatic saccharification begins with production of an organic acid by hydrolysis of hemicellulose.

3. The enzymatic saccharification method of biomass to minimize the production of metabolites by the contaminated microorganisms of claim 1, wherein the change in the pH of the enzymatic saccharification system during the biomass enzymatic saccharification processing begins with production or an organic acid by growth of microorganisms existing in the enzymatic saccharification system.

4. The enzymatic saccharification method of biomass to minimize the production of metabolites by the contaminated microorganisms of claim 1, wherein detecting the microbial contamination comprises using the interval between alkali injections due to the operation of the alkali injection pump.

5. The enzymatic saccharification method of biomass to minimize the production of metabolites by the contaminated microorganisms of claim 1, wherein, in detecting of the microbial contamination, the time taken by the enzymatic saccharification system to lower the pH to the preset lower limit after a certain amount of the alkali is injected, or the time interval between the point in time when the pH is increased by operating the alkali injection pump and the point in time when the alkali injection pump is operated again is compared with previous measurements, and the point in time point when microbial contamination substantially begins is determined to be the point in time when the current measurement is reduced by over a preset ratio in comparison with the previous measurements.

6. The enzymatic saccharification method of biomass to minimize the production of metabolites by the contaminated microorganisms of claim 1, wherein, in the detecting of the critical point in time, the time taken by the enzymatic saccharification system to lower the pH to the preset lower limit after a certain amount of the alkali is injected, or the time interval between the point in time when the pH is increased by operation of the alkali injection pump and the point in time when the alkali injection pump operates again is compared with previous measurements, and the critical point in time when the microbial contamination is too severe to continue the enzymatic saccharification processing is determined to be the time point when a current measurement is further reduced by over a preset ratio in comparison with previous measurements.

7. The enzymatic saccharification method of biomass to minimize the production of metabolites by the contaminated microorganisms of claim 1, wherein the critical point in time when the microbial contamination is too severe to continue the enzymatic saccharification processing is determined as a time point when a total amount of the alkali injected after the detecting of the microbial contamination to adjust the pH of the enzymatic saccharification system becomes equal to an organic acid equivalent allowed as a microbial metabolite.

8. The enzymatic saccharification method of biomass to minimize the production of metabolites by the contaminated microorganisms of claim 1, wherein, at the critical point in time when the microbial contamination is too severe to continue the enzymatic saccharification, the operating conditions of the enzymatic saccharification system are rapidly converted to conditions under which microbial growth is strongly inhibited by promptly adding an acid or alkali to convert the pH, at which microorganisms may no longer grow, or by rapidly cooling the enzymatic saccharification system to a temperature at which microorganisms may no longer grow.

9. A biomass saccharification reactor to minimize production of metabolites by contaminated microorganisms, the biomass saccharification reactor comprising:
an enzymatic saccharification reactor for saccharification of the biomass or biomass pretreatment products;
a pH measurement device of saccharification product for measuring pH of saccharification products in the enzymatic saccharification reactor;
a pump for providing an alkali into the enzymatic saccharification reactor;
a pH adjustment device of the enzymatic saccharification reactor for adjusting the pH in the enzymatic saccharification reactor by controlling an amount of the alkali injected by the alkali pump, or an injection interval according to the pH of the saccharification products measured by pH measurement device of the saccharification product; and
a thermostat for keeping a temperature of the saccharification reactor constant.

10. The biomass saccharification reactor to minimize the production metabolites by the contaminated microorganisms of claim 9, wherein the biomass saccharification reactor further comprises a temperature adjustment device for rapidly cooling the saccharification reactor.

11. The enzymatic saccharification method of biomass to minimize the production of metabolites by the contaminated microorganisms of claim 1, wherein detecting the microbial contamination comprises measuring the rate of pH change to determine the amount of time taken by the enzymatic saccharification system to change the pH to the preset lower limit alter adjustment by injection of the alkali and comparing the measured rate of pH change to the previous measured rate of pH change.
